(19) United States
(12) Patent Application Publication
(10) Pub. No.: US 2026/0284065 A1
(43) Pub. Date: Sep. 24, 2026

(54) PHARMACEUTICAL COMPOSITION FOR PREVENTION OR TREATMENT OF CANCER FOR OVERCOMING RESISTANCE TO IMMUNE CHECKPOINT INHIBITORS

(71) Applicant: AJOU UNIVERSITY INDUSTRY-ACADEMIC COOPERATION FOUNDATION, Suwon-si, Gyeonggi-do (KR)

(72) Inventors: Hoon HUR, Seongnam-si (KR); Tae Hoon KIM, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 19/475,270

(22) PCT Filed: Apr. 15, 2024
(86) PCT No.: PCT/KR2024/005019

§ 371 Date: Oct. 14, 2025

(30) Foreign Application Priority Data
Apr. 21, 2023 (KR) 10-2023-0052637

(51) Int. Cl. A61K 31/5377; A61K 45/06; A61P 35/00

(52) U.S. Cl. CPC A61K 31/5377; A61K 45/06; A61P 35/00

(57) ABSTRACT

The present disclosure relates to a pharmaceutical composition for the prevention or treatment of cancer capable of overcoming resistance in subjects that exhibit resistance to immune checkpoint inhibitors and enhancing anti-tumor immunity. The composition comprises an AXL kinase inhibitor, and an immune checkpoint inhibitor.

### TECHNICAL FIELD

The present disclosure relates to a pharmaceutical composition for the prevention or treatment of cancer for overcoming resistance to immune checkpoint inhibitors.

### BACKGROUND ART

Cancer is a disease having mortality rate that continues to increase every year due to environmental changes, aging population, and changes in lifestyle. Cancer is one of the top five causes of death worldwide and a disease that takes the first cause of death in Korea. Cancer treatment is performed through surgery, radiation therapy, and drug therapy. In the case of drug therapy, starting with the first-generation chemotherapy, the second-generation targeted drugs therapy has been developed, and has been currently the most widely used, but is facing a major problem called anticancer drug resistance. To overcome the anticancer drug resistance, immune checkpoint inhibitors as the third-generation anticancer drug have been developed and had reduced side effects, but still have a problem of showing limited advantages only to some cancer patients.

Therefore, in order to enhance cancer therapeutic effects of immune checkpoint inhibitors, research and development are required to overcome resistance to immune checkpoint inhibitors.

In the previous paper (Hoon Hur et al., “Molecular and Immune Profiling of Syngeneic Mouse Models Predict Response to Immune Checkpoint Inhibitors in Gastric Cancer,” Cancer Res Treat. 2023; 55 (1): 167-178), the present inventors prepared syngeneic mouse models using mouse gastric cancer cell lines YTN3 and YTN16 and reported that responses to immune checkpoint inhibitors varied depending on a difference in intratumoral immune cell accumulation and a difference in gene expression, and then completed the present disclosure relating to a composition for overcoming resistance to immune checkpoint inhibitors based on the results of the paper.

### DISCLOSURE

### Technical Problem

In order to solve the problems of the prior art, an object of the present disclosure is to provide a pharmaceutical composition for the prevention or treatment of cancer for overcoming resistance to immune checkpoint inhibitors.

In addition, another aspect of the present disclosure is to provide a pharmaceutical composition for overcoming resistance to immune checkpoint inhibitors.

However, objects of the present disclosure to be achieved are not limited to the above-mentioned objects, and other objects may be present.

### Technical Solution

As a technical means for achieving the above-described object, a first aspect of the present disclosure provides a pharmaceutical composition for the prevention or treatment of cancer, including an AXL kinase inhibitor and an immune checkpoint inhibitor.

According to an embodiment of the present disclosure, the AXL kinase inhibitor may include the following compound, but is not limited thereto.

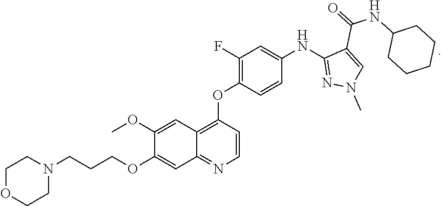 [SMILES: COc1cc2c(Oc3ccc(Nc4nn(C)cc4C(=O)NC4CCCCC4)cc3F)ccnc2cc1OCCCN1CCOCC1]

According to one embodiment of the present disclosure, the composition may increase CD8⁺ T cells and CD4⁺ T cells in a subject exhibiting resistance to the immune checkpoint inhibitor by the AXL kinase inhibitor, but is not limited thereto.

According to one embodiment of the present disclosure, the immune checkpoint inhibitor may be selected from the group consisting of anti-PD-1, anti-PD-L1, anti-CTLA4, anti-LAG-3, anti-TIM-3, and combinations thereof, but is not limited thereto.

According to one embodiment of the present disclosure, the cancer may be selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof, but is not limited thereto.

According to one embodiment of the present disclosure, the cancer may be gastric cancer, but is not limited thereto.

According to one embodiment of the present disclosure, the AXL kinase inhibitor and the immune checkpoint inhibitor may be co-administered simultaneously, separately, or sequentially, but are not limited thereto.

According to one embodiment of the present disclosure, the AXL kinase inhibitor and the immune checkpoint inhibitor may be administered each independently by a method selected from the group consisting of oral administration, intraperitoneal administration, inhalation administration, intravenous administration, intramuscular administration, subcutaneous administration, dermal administration, intrauterine administration, tumor administration, rectal administration, and combinations thereof, but are not limited thereto.

According to one embodiment of the present disclosure, the AXL kinase inhibitor and the immune checkpoint inhibitor may be administered each independently in a form selected from the group consisting of tablets, powders, granules, pills, suspensions, emulsions, infusions, capsules, syrups, injections, liquids, elixirs, extracts, links, fluid extracts, and combinations thereof, but are not limited thereto.

In addition, a second aspect of the present disclosure provides a pharmaceutical composition for overcoming resistance to immune checkpoint inhibitors, including an AXL kinase inhibitor represented by the following Chemical Formula as an active ingredient:

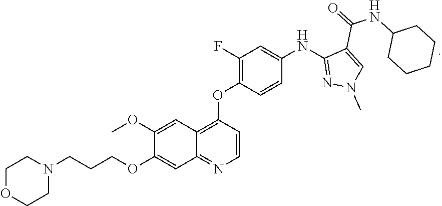 [SMILES: COc1cc2c(Oc3ccc(Nc4nn(C)cc4C(=O)NC4CCCCC4)cc3F)ccnc2cc1OCCCN1CCOCC1]

The above-mentioned technical solutions are merely illustrative and should not be construed as limiting the present disclosure. In addition to the above-described examples, additional examples may exist in the drawings and the detailed description of the present disclosure.

### Advantageous Effects

According to the present disclosure, the pharmaceutical composition for the prevention or treatment of cancer uses an AXL kinase inhibitor together with an immune checkpoint inhibitor to overcome resistance to immune checkpoint inhibitors by the AXL kinase inhibitor in subjects exhibiting the resistance to immune checkpoint inhibitors, thereby enhancing a therapeutic effect.

In addition, it can be expected that combination therapy can be applied to enhance the effect of the immune checkpoint inhibitors on patients with high AXL expression of tumors in actual clinical practice.

However, effects obtainable herein are not limited to the effects described above, and other effects may be present.

### DESCRIPTION OF DRAWINGS

FIG. 1 shows images of subcutaneous tumors in YTN16 cell lines of YTN16 syngeneic mice according to one Experimental Example of the present disclosure.

FIG. 2 shows a graph of measuring tumor weights in YTN16 syngeneic mice according to one Experimental Example of the present disclosure.

FIG. 3 shows results of observing the histological characteristics of tumors in syngeneic mice according to one Experimental Example of the present disclosure.

FIG. 4 shows results of measuring the histological characteristics of syngeneic mice according to one Experimental Example of the present disclosure.

### BEST MODE

Hereinafter, examples of the present disclosure will be described in detail so as to be easily implemented by those skilled in the art, with reference to the accompanying drawings. However, the present disclosure may be embodied in many different forms and are not limited to the examples to be described herein. In addition, in order to clearly describe the present disclosure in the drawings, parts not related with the description have been omitted, and throughout the present specification, like reference numerals designate like elements.

Further, throughout the present specification, when a certain part is “connected” with the other part, it is meant that the certain part may be “directly connected” with the other part and “electrically connected” with the other part with another element interposed therebetween.

Throughout the present specification, it will be understood that when a certain member is located “on”, “above”, “at the top of”, “under”, “below”, and “at the bottom of” the other member, the certain member is not only in contact with the other member, but also another member is present between the two members.

Throughout the present specification, when a certain part “includes” a certain element, it is meant to further include other components without excluding other components unless explicitly described to the contrary.

The terms “about”, “substantially”, and the like to be used in the present disclosure are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present disclosure. In addition, throughout the present specification, the term of “step to” or “step of” does not mean “step for”.

Throughout the present specification, the term “combinations thereof” included in the expression of the Markush form means one or more mixtures or combinations selected from the group consisting of components described in the expression of the Markush form, and means to include at least one selected from the group consisting of the components.

Throughout the present specification, “A and/or B” means “A or B, or A and B”.

Hereinafter, a composition for the prevention or treatment of cancer for overcoming resistance to immune checkpoint inhibitors of the present disclosure and a pharmaceutical composition for overcoming resistance to immune checkpoint inhibitors will be described in detail with reference to embodiments, Examples, and drawings. However, the present disclosure is not limited to these embodiments, Examples, and drawings.

As a technical means for achieving the above-described object, the first aspect of the present disclosure provides a pharmaceutical composition for the prevention or treatment of cancer, including an AXL kinase inhibitor and an immune checkpoint inhibitor.

The immune checkpoint inhibitor is a type of drug that blocks proteins called immune checkpoints expressed in some immune cells, such as T cells, and cancer cells, in a form of cancer immunotherapy, and has attracted attention as a therapeutic agent with fewer side effects and more improved therapeutic effects than existing anticancer agents. However, there was a problem that the immune checkpoint inhibitor did not exhibit the therapeutic effect in most patients, but exhibited the therapeutic effect only in some patients.

However, the pharmaceutical composition for the prevention or treatment of cancer according to the present disclosure uses an AXL kinase inhibitor together with the immune checkpoint inhibitor to overcome resistance to the immune checkpoint inhibitor by the AXL kinase inhibitor in a subject exhibiting resistance to the immune checkpoint inhibitor, thereby enhancing a therapeutic effect.

In addition, it may be expected that combination therapy may be applied to enhance the effect of the immune checkpoint inhibitors on patients with high AXL expression of tumors in actual clinical practice.

According to an embodiment of the present disclosure, the AXL kinase inhibitor may include the following compound, but is not limited thereto.

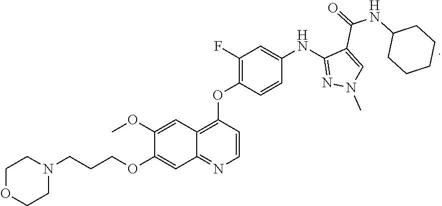 [SMILES: COc1cc2c(Oc3ccc(Nc4nn(C)cc4C(=O)NC4CCCCC4)cc3F)ccnc2cc1OCCCN1CCOCC1]

According to one embodiment of the present disclosure, the immune checkpoint inhibitor may be selected from the group consisting of anti-PD-1, anti-PD-L1, anti-CTLA4, anti-LAG-3, anti-TIM-3, and combinations thereof, but is not limited thereto.

According to one embodiment of the present disclosure, the composition may increase CD8⁺ T cells and CD4⁺ T cells in a subject exhibiting resistance to the immune checkpoint inhibitor by the AXL kinase inhibitor, but is not limited thereto.

The present inventors prepared an syngeneic mouse model using a mouse gastric cancer cell line having resistance to the immune checkpoint inhibitor, and confirmed that a group administered to the mouse model with the AXL kinase inhibitor together with the immune checkpoint inhibitor showed the greatest therapeutic effect. Specifically, the smallest tumor size was shown in the group, and it was confirmed that among immune cells infiltrating the tumor, CD8⁺ T cells (CD8a) and CD4⁺ T cells, which increased anti-tumor immunity, were increased.

According to one embodiment of the present disclosure, the cancer may be selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof, but is not limited thereto.

According to an embodiment of the present disclosure, the cancer may be gastric cancer, but is not limited thereto.

According to one embodiment of the present disclosure, the AXL kinase inhibitor and the immune checkpoint inhibitor may be co-administered simultaneously, separately, or sequentially, but are not limited thereto.

According to one embodiment of the present disclosure, the AXL kinase inhibitor and the immune checkpoint inhibitor may be administered each independently by a method selected from the group consisting of oral administration, intraperitoneal administration, inhalation administration, intravenous administration, intramuscular administration, subcutaneous administration, dermal administration, intrauterine administration, tumor administration, rectal administration, and combinations thereof, but are not limited thereto.

It is preferable to use an administration route of the pharmaceutical composition for the prevention or treatment of cancer effective for the prevention or treatment of cancer according to one embodiment of the present disclosure, and the AXL kinase inhibitor and the immune checkpoint inhibitor, which are active ingredients included in the composition, may be administered by the same administration route or by different routes.

According to one embodiment of the present disclosure, the AXL kinase inhibitor and the immune checkpoint inhibitor may be administered each independently in a form selected from the group consisting of tablets, powders, granules, pills, suspensions, emulsions, infusions, capsules, syrups, injections, liquids, elixirs, extracts, links, fluid extracts, and combinations thereof, but are not limited thereto.

The oral administration may include a preparation selected from the group consisting of syrups, tablets, powders, granules, and combinations thereof, but is not limited thereto.

The syrups may include materials selected from the group consisting of diluents, such as water, sugars such as sucrose, sorbitol, and fructose, glycols such as polyethylene glycol and propylene glycol, oils such as sesame oil, olive oil, and soybean oil, etc.; preservatives such as p-hydroxybenzoic acid esters, etc.; parahydroxybenzoic acid derivatives such as methyl parahydroxybenzoate, etc.; preservatives such as sodium benzoate, etc.; flavorings such as strawberry flavor, peppermint, etc.; and combinations thereof, but are not limited thereto.

The tablets, powders and granules may include, but are not limited to, materials selected from the group consisting of sugars such as lactose, white sugar, glucose, sucrose, mannitol and sorbitol; starches such as potatoes, wheat and corn; inorganic materials such as calcium carbonate, calcium sulfate, sodium bicarbonate and sodium chloride; excipients such as crystalline cellulose and plant powders such as licorice powder and gentian powder; disintegrants such as starch, agar, gelatin powder, crystalline cellulose, sodium carmellose, calcium carmellose, calcium carbonate, sodium bicarbonate and sodium alginate; lubricants such as magnesium stearate, talc, hydrogenated vegetable oils, macrogol and silicone oil; binders such as polyvinyl alcohol, hydroxypropyl cellulose, methylcellulose, ethylcellulose, carmellose, gelatin and starch paste; surfactants such as fatty acid esters; plasticizers such as glycerin; and combinations thereof.

A preparation suitable for the intraperitoneal administration, intravenous administration, intramuscular administration, subcutaneous administration, dermal administration, intrauterine administration, tumor administration, and rectal administration may consist of a sterile aqueous solution containing an active compound which is isotonic with a recipient's blood, but is not limited thereto. For example, in the case of injections, an injectable solution may be prepared using carriers consisting of a salt solution, a glucose solution, or a mixture of saline and glucose solutions, and the like, but is not limited thereto.

The preparation suitable for the intraperitoneal administration, intravenous administration, intramuscular administration, subcutaneous administration, dermal administration, intrauterine administration, tumor administration, and rectal administration may include a material selected from the group consisting of a diluent, an antiseptic, a preservative, a fragrance, an excipient, a disintegrant, a lubricant, a binder, a surfactant, a plasticizer, and combinations thereof, but is not limited thereto.

In addition, a second aspect of the present disclosure provides a pharmaceutical composition for overcoming resistance to immune checkpoint inhibitors, including an AXL kinase inhibitor represented by the following Chemical Formula as an active ingredient:

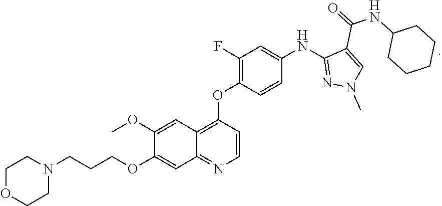 [SMILES: COc1cc2c(Oc3ccc(Nc4nn(C)cc4C(=O)NC4CCCCC4)cc3F)ccnc2cc1OCCCN1CCOCC1]

With respect to the pharmaceutical composition for overcoming resistance to immune checkpoint inhibitors according to the second aspect of the present disclosure, parts overlapping with those of the first aspect of the present disclosure have been omitted in the detailed description, but even if the description thereof had been omitted, the contents described in the first aspect of the present disclosure may be equally applied to the second aspect of the present disclosure.

The pharmaceutical composition for overcoming the resistance to immune checkpoint inhibitors according to the present disclosure may be administered together with an immune checkpoint inhibitor to a subject exhibiting the resistance to the immune checkpoint inhibitor, thereby overcoming the resistance to the immune checkpoint inhibitor and enhancing a therapeutic effect.

Hereinafter, the present disclosure will be described in more detail with reference to the following Examples, but the following Examples are only for illustrative purposes and are not intended to limit the scope of the present disclosure.

### [Example] Anti-PD-1+CCB-3233

A composition including an immune checkpoint inhibitor Anti-PD1 and an AXL kinase inhibitor CCB-3233 represented by the following Chemical Formula was used as Example:

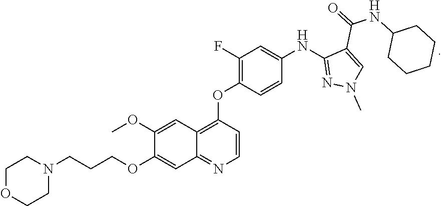 [SMILES: COc1cc2c(Oc3ccc(Nc4nn(C)cc4C(=O)NC4CCCCC4)cc3F)ccnc2cc1OCCCN1CCOCC1]

### [Comparative Example 1] Anti-PD-1

A composition including only the immune checkpoint inhibitor Anti-PD1 used in Example was used as Comparative Example 1.

### [Comparative Example 2] CCB-3233

A composition including only the AXL kinase inhibitor CCB-3233 used in Example was used as Comparative Example 2.

### Experimental Example 1

Based on results of the previous study (Hoon Hur et al, “Molecular and Immune Profiling of Syngeneic Mouse Models Predict Response to Immune Checkpoint Inhibitors in Gastric Cancer,” Cancer Res Treat. 2023; 55 (1): 167-178), the present inventors conducted an experiment to determine an effect of the composition according to Example in syngeneic mouse models using a mouse gastric cancer cell line YTN16, which exhibited resistance to immune checkpoint inhibitors.

The experiment was conducted by dividing the mice into a control group (Control), a group administered with Comparative Example 1 (Anti-PD-1), a group administered with Comparative Example 2 (CCB-3233), and a group administered with Example 1 (Anti-PD-1+CCB-3233), and eight mouse models were used for each group.

Comparative Example 1 (Anti-PD-1) was diluted in 1×PBS at a concentration of 10 mg/kg and administered intraperitoneally to the mice, Comparative Example 2 (CCB-3233) was orally administered to the mice at a concentration of 50 mg/kg by dissolving 5% DMSO+5% CMC-NA in 1×PBS, and Example 1 (Anti-PD-1+CCB-3233) was diluted and prepared in different solvents as in Comparative Examples 1 and 2, and then administered sequentially.

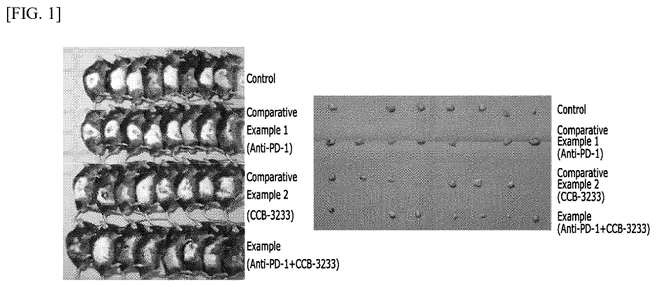

FIG. 1 shows images of subcutaneous tumors in YTN16 cell lines of YTN16 syngeneic mice according to one Experimental Example of the present disclosure.

Referring to FIG. 1, it was visually confirmed that the group administered with the composition according to Example of the present disclosure had the smallest tumor size.

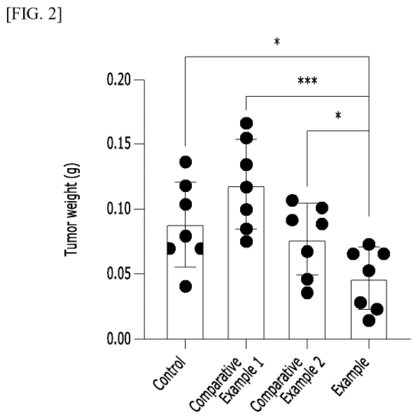

FIG. 2 shows a graph of measuring tumor weights in YTN16 syngeneic mice according to one Experimental Example of the present disclosure.

Referring to FIG. 2, it was confirmed that the group administered with Comparative Example 1 (anti-PD-1) had the highest tumor weight, while it was confirmed that the group administered with Comparative Example 2 (CCB-3233) had no significant difference in tumor weight from the control group.

In contrast, the group administered with Example 1 (anti-PD-1+CCB-3233) had the smallest tumor weight, and as a result, it could be confirmed that the AXL inhibitor and the immune checkpoint inhibitor were simultaneously used to enhance therapeutic effect.

### Experimental Example 2

An experiment was conducted to analyze the histological characteristics of the tumors of the syngeneic mice used in Experimental Example 1.

Syngeneic mouse models were constructed using mouse gastric cancer cell lines, YTN3 and YTN16 cells, and tumors generated from each cell were subjected to H&E tissue staining and immunohistochemical staining, and then the histological characteristics were classified by a pathologist.

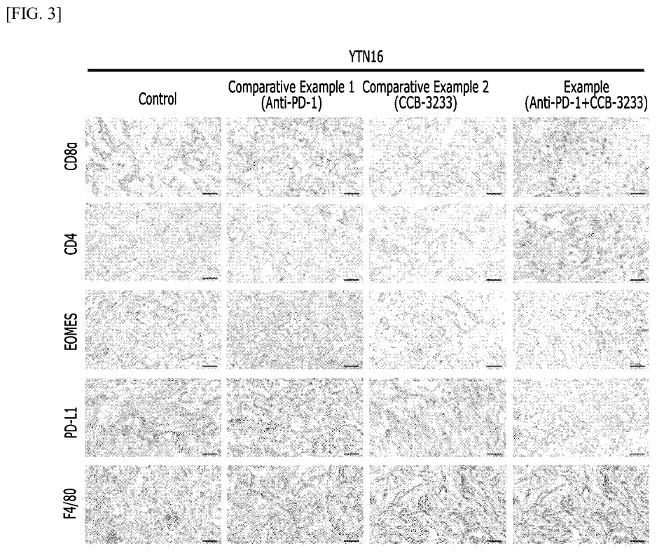

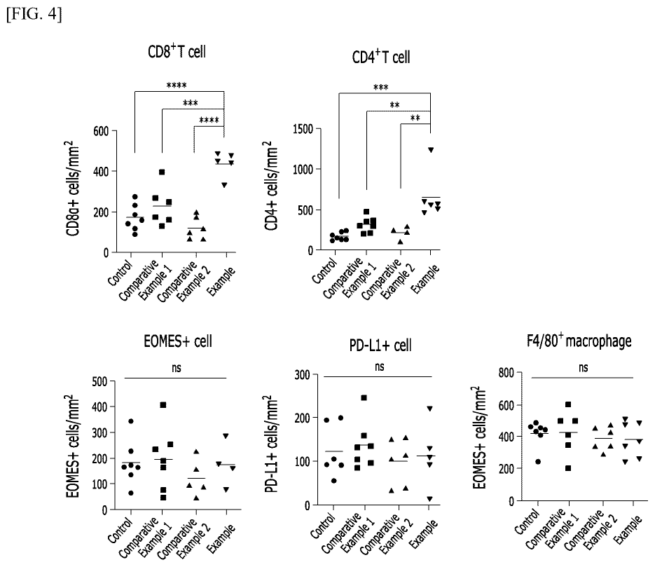

FIG. 3 shows results of observing the histological characteristics of tumors of syngeneic mice according to one Experimental Example of the present disclosure, and FIG. 4 shows results of measuring the histological characteristics of the syngeneic mice according to one Experimental Example of the present disclosure.

Referring to FIG. 3, in the case of tumors isolated from the group administered with Example 1 (anti-PD-1+CCB-3233), it was confirmed through immunohistochemical staining that among immune cells infiltrating the tumors, CD8α and CD4 T cells, which enhance anti-tumor immunity, were increased.

Referring to FIG. 4, in the case of tumors isolated from the group administered with Example 1 (anti-PD-1+CCB-3233), it was confirmed through flow cytometry that among immune cells infiltrating the tumors, CD8α and CD4 T cells, which enhanced anti-tumor immunity, were increased, as the results shown in FIG. 3.

Through Experimental Examples 1 and 2, it was confirmed that when administering the AXL kinase inhibitor CCB-3233 together with the immune checkpoint inhibitor to YTN 16 syngeneic mice having resistance to the immune checkpoint inhibitor, the therapeutic effect of the immune checkpoint inhibitor was enhanced, and as a result, it was confirmed that CCB-3233 could be used as a drug to overcome resistance to the immune checkpoint inhibitor.

The aforementioned description of the present disclosure is to be exemplified, and it will be understood by those skilled in the art that the present disclosure may be easily modified in other detailed forms without changing the technical spirit or required features of the present disclosure. Therefore, it should be appreciated that the examples described above are illustrative in all aspects and are not restricted. For example, each component described as a single form may be implemented in a distributed form, and similarly, components described as distributed form may be implemented in a combined form.

The scope of the present disclosure is represented by appended claims to be described below rather than the detailed description, and it is to be interpreted that the meaning and scope of the claims and all the changes or modified forms derived from the equivalents thereof come within the scope of the present disclosure.

1. A pharmaceutical composition for prevention or treatment of cancer, comprising:
an AXL Kinase Inhibitor; and
an immune checkpoint inhibitor.

2. The pharmaceutical composition for prevention or treatment of cancer of claim 1, wherein the AXL kinase inhibitor comprises the following compound:

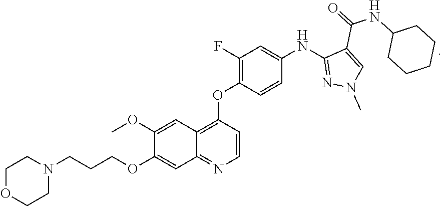 [SMILES: COc1cc2c(Oc3ccc(Nc4nn(C)cc4C(=O)NC4CCCCC4)cc3F)ccnc2cc1OCCCN1CCOCC1]

3. The pharmaceutical composition for prevention or treatment of cancer of claim 2, wherein the composition increases CD8⁺ T cells and CD4⁺ T cells in a subject exhibiting resistance to the immune checkpoint inhibitor by the AXL kinase inhibitor.

4. The pharmaceutical composition for prevention or treatment of cancer of claim 1, wherein the immune checkpoint inhibitor is selected from the group consisting of anti-PD-1, anti-PD-L1, anti-CTLA4, anti-LAG-3, anti-TIM-3, and combinations thereof.

5. The pharmaceutical composition for prevention or treatment of cancer of claim 1, wherein the cancer is selected from the group consisting of gastric cancer, esophageal cancer, pancreatic cancer, breast cancer, melanoma, thyroid cancer, liver cancer, glioblastoma, ovarian cancer, colon cancer, head and neck cancer, bladder cancer, renal cell cancer, metastatic cancer, prostate cancer, lung cancer and combinations thereof.

6. The pharmaceutical composition for prevention or treatment of cancer of claim 5, wherein the cancer is gastric cancer.

7. The pharmaceutical composition for prevention or treatment of cancer of claim 1, wherein the AXL kinase inhibitor and the immune checkpoint inhibitor are co-administered simultaneously, separately, or sequentially.

8. The pharmaceutical composition for prevention or treatment of cancer of claim 7, wherein the AXL kinase inhibitor and the immune checkpoint inhibitor are administered each independently by a method selected from the group consisting of oral administration, intraperitoneal administration, inhalation administration, intravenous administration, intramuscular administration, subcutaneous administration, dermal administration, intrauterine administration, tumor administration, rectal administration, and combinations thereof.

9. The pharmaceutical composition for prevention or treatment of cancer of claim 7, wherein the AXL kinase inhibitor and the immune checkpoint inhibitor are administered each independently in a form selected from the group consisting of tablets, powders, granules, pills, suspensions, emulsions, infusions, capsules, syrups, injections, liquids, elixirs, extracts, links, fluid extracts, and combinations thereof.

10. A pharmaceutical composition for overcoming resistance to immune checkpoint inhibitors, comprising an AXL kinase inhibitor represented by the following Chemical Formula as an active ingredient: 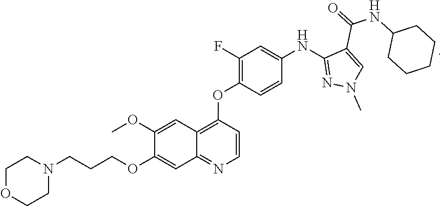 [SMILES: COc1cc2c(Oc3ccc(Nc4nn(C)cc4C(=O)NC4CCCCC4)cc3F)ccnc2cc1OCCCN1CCOCC1]
